(19) United States
(12) Patent Application Publication
(10) Pub. No.: US 2012/0316541 A1
(43) Pub. Date: Dec. 13, 2012

(54) DUAL-PURPOSE LAPAROSCOPIC SURGICAL DEVICE

(76) Inventors: Sarah J. Young, Menlo Park, CA (US); Pablo Garcia, Menlo Park, CA (US); Karen Shakespear, San Francisco, CA (US); Sanjeev Dutta, Los Altos, CA (US); Susan Kim, Menlo Park, CA (US)

(21) Appl. No.: 13/491,030

(22) Filed: Jun. 7, 2012

Related U.S. Application Data
(60) Provisional application No. 61/520,273, filed on Jun. 7, 2011.

(51) Int. Cl. A61B 17/00

(52) U.S. Cl. 606/1

(57) ABSTRACT

A medical device intended for use in performing minimally invasive procedures is provided. The device is intended for performing a pyloromyotomy in pediatric surgery, laparascopically. A key aspect of embodiments of the device is the dual-purpose functionality of the device as it combines both spreading of tissue and cutting of tissue, using mono- or bi-polar electric energy or a knife, with the same medical device. The use of the device requires fewer tool changes during surgery, improving safety and ease of use, as well as reducing surgery time. The devices lend themselves to torelatively cheaper manufacturing processes and lower overall cost compared to existing devices. They would also require less training time compared to current laparoscopic tools.

### CROSS-REFERENCE TO RELATED APPLICATIONS

This application claims priority from U.S. Provisional Patent Application 61/520,273 filed Jun. 7, 2011, which is incorporated herein by reference.

### STATEMENT OF GOVERNMENT SPONSORED SUPPORT

This invention was made with Government support under contract 1P50FD003782-01 awarded by Department of Health & Human Services. The Government has certain rights in this invention.

### FIELD OF THE INVENTION

This invention relates generally to medical devices. In particular, the invention relates to laparoscopic devices and procedures.

### BACKGROUND OF THE INVENTION

Pyloromyotomy is a surgical procedure in which an incision is made in the longitudinal and circular muscles of the pylorus. It is used to treat hypertrophic pyloric stenosis. Current surgical tools for pyloromyotomies are not adequate and require extensive training The present invention addresses these problems.

### SUMMARY OF THE INVENTION

This invention provides a medical device intended for use in performing minimally invasive procedures. In one specific embodiment, the device is intended for performing a pyloromyotomy in pediatric surgery, laparascopically. A key aspect of embodiments of the device is the dual-purpose functionality of the device as it combines both spreading of tissue and cutting of tissue, using mono- or bi-polar electric energy or a knife, with the same medical device. In the monopolar case, the return electrode would be a Patient Return Pad. In the bipolar case both electrodes are present in the device itself and no return pad is required.

In the example of pyloromyotomy, the device cuts tissue and also spreads the two sides of the incision apart to allow for careful dissection down to the mucosa of the pylorus. The use of the device according to this invention requires fewer tool changes during surgery, improving safety and ease of use, as well as reducing surgery time. The embodiments described herein lend themselves to torelatively cheaper manufacturing processes and lower overall cost as compared to existing devices. They would also require less training time compared to current laparoscopic tools.

In another embodiment, the device is intended for performing minimally invasive, laparascopic or endoscopic procedures requiring grasping, cautery, and/or cutting for other clinical areas, including, but not only limited to, ENT, neurosurgery, neuroendoscopy, orthopedics, gasteroenterology and urology.

The device can be used with a stabilizer device to hold and control the pyloris during the operation.

### BRIEF DESCRIPTION OF THE DRAWINGS

FIG. 1 shows a tip design according to an exemplary embodiment of the invention.

FIG. 2 shows a tip design according to another exemplary embodiment of the invention.

FIG. 3 shows a bipolar version of the tool tip according to an exemplary embodiment of the invention.

FIG. 4 shows a tip design according to yet another exemplary embodiment of the invention.

FIG. 5 shows a handle design according to an exemplary embodiment of the invention.

FIG. 6 shows a handle design according to another exemplary embodiment of the invention.

FIGS. 7A-B show a tip design for the bladder actuated tip tool according to an exemplary embodiment of the invention. FIG. 7A shows a closed position and FIG. 7B shows an open position.

FIGS. 8A-B show a tip design for the bladder actuated tip tool according to another exemplary embodiment of the invention. FIG. 8A shows a closed position and FIG. 8B shows an open position.

### DETAILED DESCRIPTION

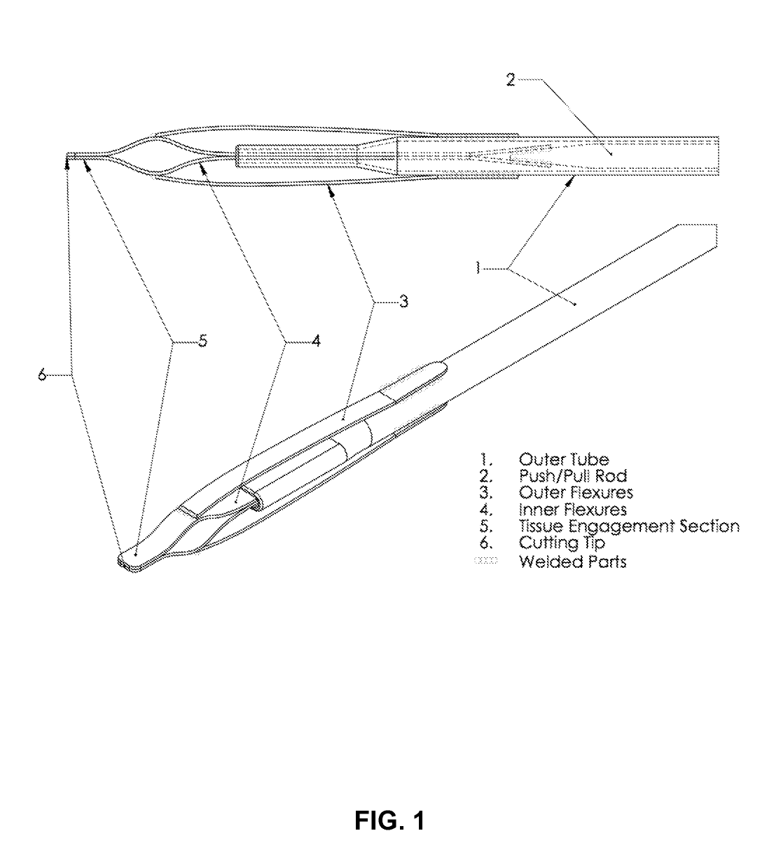

FIG. 1 shows an exemplary embodiment of tip design according to invention. The tip is made from one Outer Tube 1 that is compressed slightly at the distal end, one Rod 2 that is chamfered at the distal end, and four strips of flexible sheet metal in the form of two Inner Flexures 3 and two Outer Flexures 4. These parts are joined as indicated by welds. The tip is entirely coated in a thin insulating material except for the Cutting Tip 6. With reference to FIG. 1, the tip functions in the following manner. A small incision is made in the tissue using the Cutting Tip 6, which is an electrically active area that cuts in a manner similar to other electrosurgical tools. The energy required to cut is less than that of current tools because the area of the Cutting Tip 6 is smaller. Next, the most distal portion of the tip 5 is inserted into the incision in the tissue at the specific location where tissue spreading is desired. When the handle is squeezed, the Push/Pull Rod 2 is pushed toward the tip while the Outer Tube 1 is held stationary, which causes the Inner Flexures 4 to separate from each other. This pressure pushes the edges of the incision apart, spreading the tissue. The Tissue Engagement Section 5 may or may not have features such as ridges or punches that aid in holding the tissue while it is being spread apart.

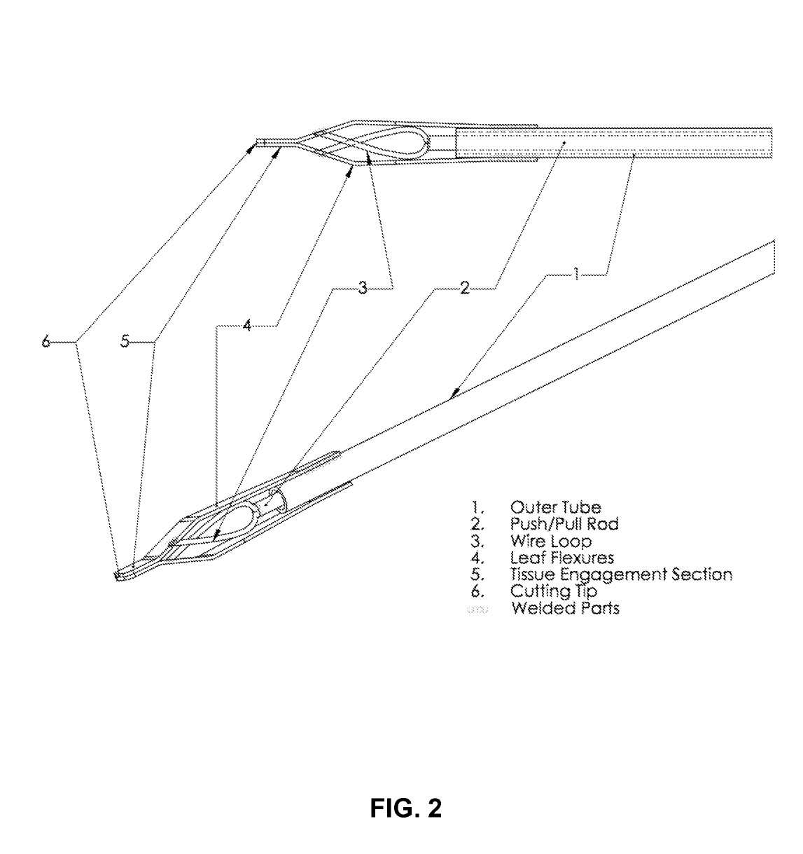

FIG. 2 shows another exemplary embodiment of tip design according to invention. The tip is made from one Outer Tube 1, one Push/Pull Rod 2, two strips of flexible sheet metal shaped into Leaf Flexures 4, and one loop of flexible wire 3. These parts are joined as indicated by welds. The tip is entirely coated in a thin insulating material except for the Cutting Tip 6. With reference to FIG. 2, the tip functions in the manner as described with respect to FIG. 1, with the exception that the Inner Flexures (4 in FIG. 1) are replaced by the Wire Loop (3 in FIG. 2).

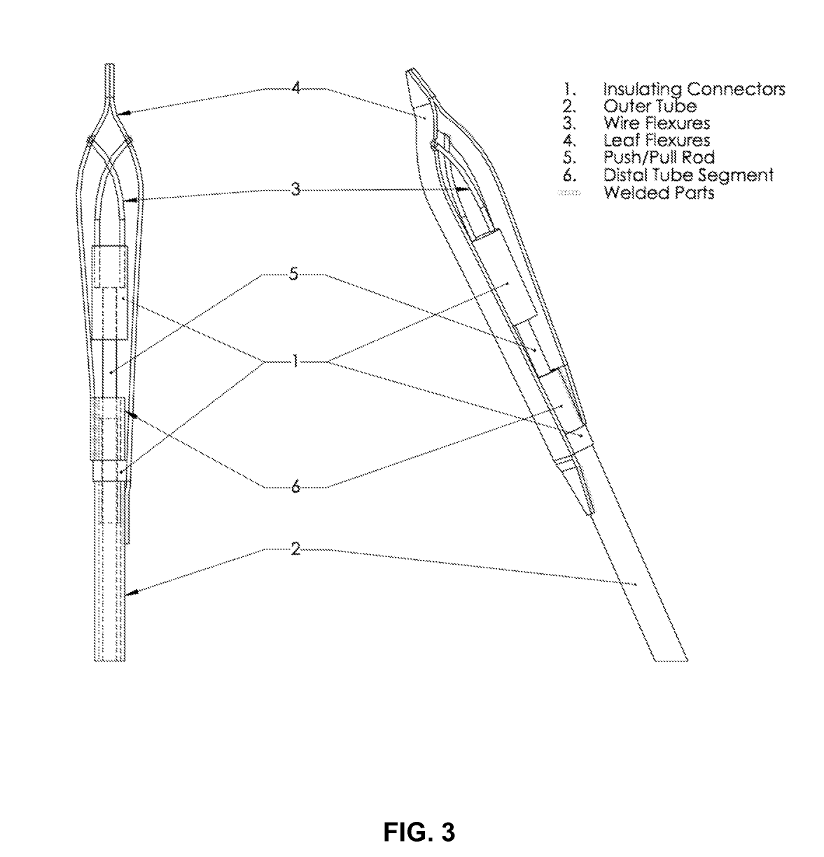

FIG. 3 shows an exemplary embodiment for a bipolar version of the tool tip according to invention. In the bipolar embodiment, the tip would be configured to operate as a grasper instead of a spreader. This tip, like the wire loop tip shown in

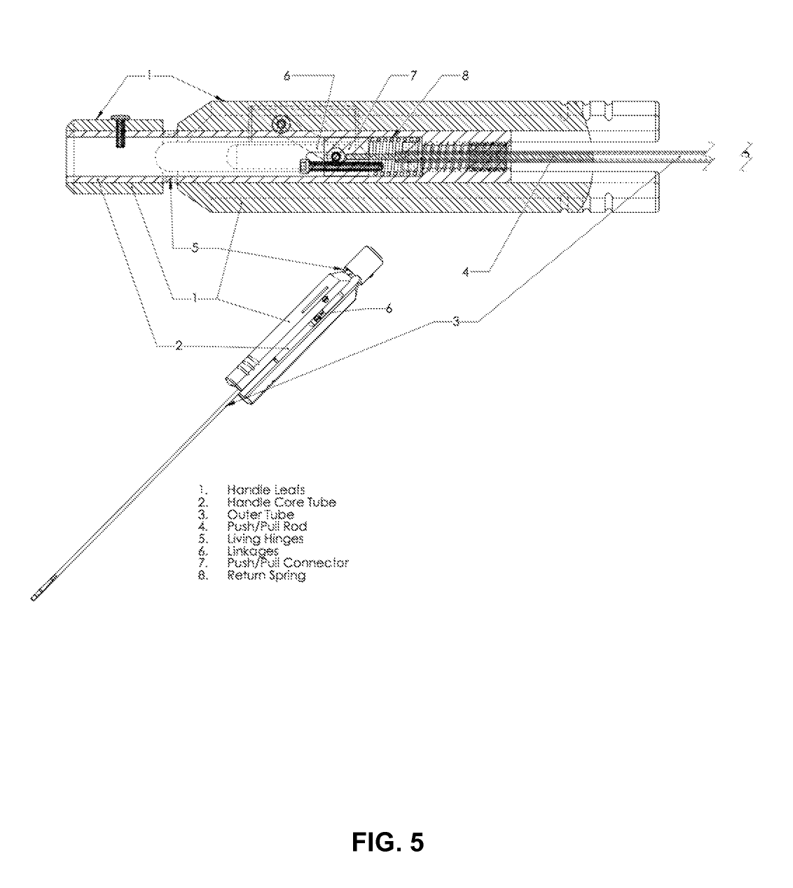

FIG. 2, is composed of an Outer Tube 2, a Push/Pull Rod 5 and two strips of flexible sheet metal shaped into Leaf Flexures 4. The wire loop of FIG. 2 is replaced by two Wire Flexures 3 in this embodiment. The notable difference of this tip design is that the two Leaf Flexures 4 are electrically isolated from each other when they are spread apart by the Push/Pull Rod. This is possible due to the use of Insulating Connectors 1 that join the Outer Tube 2 to the Distal Tube Segment 6 as well as the Push/Pull Rod 5 and the Wire Flexures 3. These connectors are made out of an insulating material such as a polymer. The metal components that they hold together may be joined to the connectors by adhesive. The metal components are joined together by welds as indicated in FIG. 3. The entire tip is coated in a thin insulating coating except for the areas on the inside of the Leaf Flexures (shown in contact with each other in FIG. 3). The tip functions in the following manner. Unlike in FIGS. 1-2, the tip starts out in the “open” position with the Leaf Flexures spread apart. With the Leaf Flexures in the spread position, the tool is moved into position with the flexures around the tissue to be cauterized. The Leaf Flexures are then closed around the tissue and the bipolar electrical energy is activated, cauterizing the tissue between the two Leaf Flexures. The bipolar tip version could be paired with a “grasper” handle style (see e.g. FIG. 5 description).

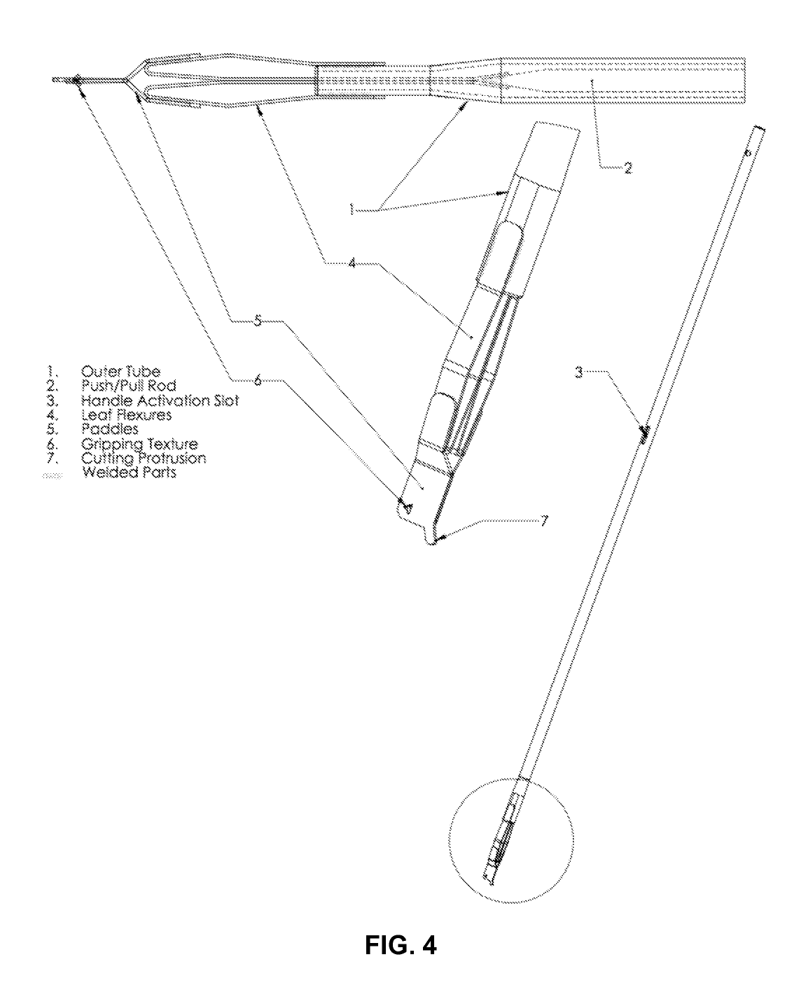

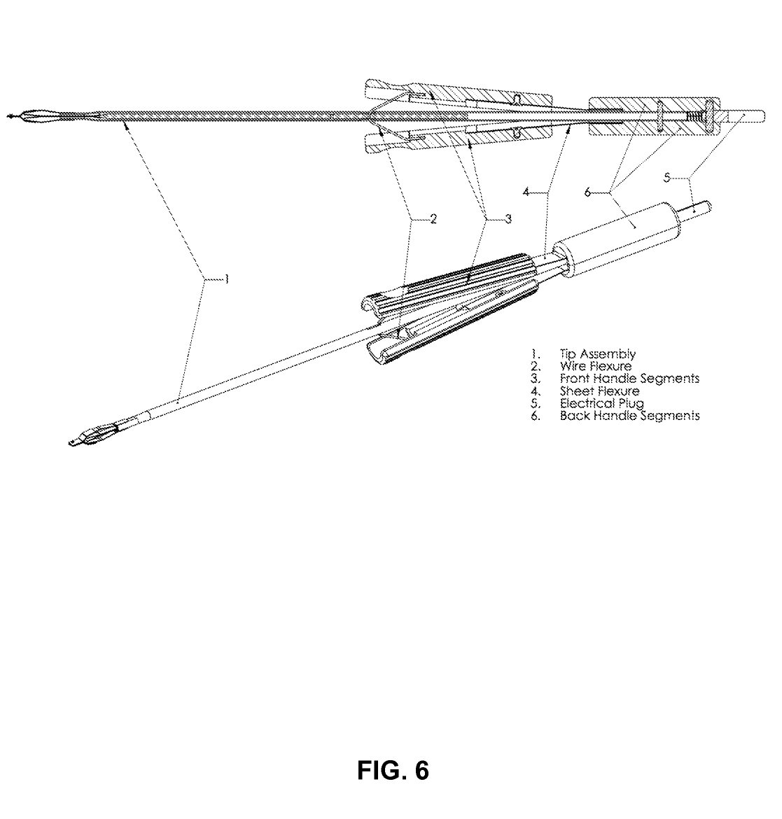

FIG. 4 shows another exemplary embodiment of a tip design according to invention. As in FIG. 2, the tip is made from one Outer Tube 1, one Push/Pull Rod 2 and two strips of flexible sheet metal shaped into Leaf Flexures 4. In this embodiment, however, the Leaf Flexures are bent 180 degrees back on themselves so that they can be attached directly to the Push/Pull Rod. Additionally, two Paddles 5 are attached to the Leaf Flexures to create a thin distal tip for easy insertion into the tissue. FIG. 4 also shows an example of a Gripping Texture 6 on the distal portion of the tip that is used to engage with the tissue and prevent slipping while using the spreading feature. A Cutting Protrusion 7 is also shown. All areas of the tip could be coated in a thin insulating coating except for this Cutting Protrusion. This could enable the very small area of the protrusion to be the only electrically active cutting portion of the tip, decreasing the energy required to cut tissue as compared to devices with a larger electrically active area. The Handle Activation Slot 3 has a slot in the Outer Tube 1 and a small hole in the Push/Pull Rod 2 that allows the tip to interface with the Wire Flexure of the handle (see FIG. 6). The tip is assembled with welds as indicated and functions in the same manner as the tip described in FIG. 1.

FIG. 5 shows an exemplary embodiment of a handle design according to invention. The handle is made from a Handle Core Tube 2 and a Handle Leafs part 1 along with Linkages 6, a Push/Pull Connector 7 and other mechanical components that join the handle to the Outer Tube 3 and inner Push/Pull Rod 4 of the tip. The Living Hinges 5 of the Handle Leafs part 1 allow the leafs to flex open and closed. They are shown in FIG. 5 in the “closed” position; in the “open” position they are separated from the inner Handle Core Tube 2. When the leafs are squeezed closed, the Linkages 6 push the Push/Pull Connector 7 towards the distal tip of the tool, which moves the Push/Pull Rod 4 and causes the tip to spread. When pressure is released from the leafs, an internal spring in the Handle Core Tube 2 pushes the Push/Pull Connector 7 back to its original position, closing the tip of the tool. To make the tip a “grasper” instead of a “spreader” (normally open instead of normally closed), this handle design can be modified in the following ways. The Linkages 6 would be flipped such that the joint between the Linkage and the Handle Leaf was distal to the joint between the Linkage and the Push/Pull Connector. Additionally, the return spring could be positioned on the proximal side of the Push/Pull Rod Connector instead of on the distal side as shown.

FIG. 6 shows another exemplary embodiment of a handle design according to invention. This handle differs from the handle in FIG. 5 in that it uses flexures instead of linkages to move the Push/Pull Rod and activate spreading of the tip. The handle in FIG. 6 is composed of two Front Handle Segments 3 and two Back Handle Segments 6. They are joined by a Sheet Flexure 4, and the Back Handle Segments are pinned together. The Back Handle Segments hold the Outer Tube of the tip steady, while squeezing the Front Handle Segments causes the Wire Flexure 2 to push the Push/Pull Rod forward and activate the spreading of the tip. Releasing pressure on the Front Handle Segments allows the flexures to return to their natural shape, pulling the Push/Pull Rod back and closing the tip.

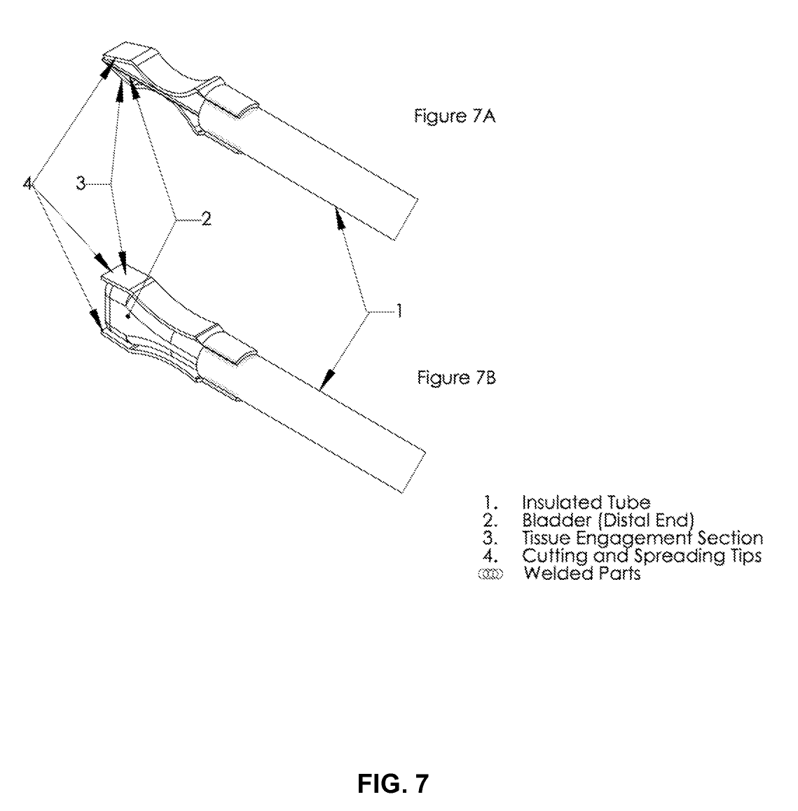

FIGS. 7A-B show another exemplary embodiment of a tip design for the bladder actuated tip tool according to invention. The Cutting and Spreading Tips 4 are welded on to a tube, which is then insulated 1. In the closed position, the fluid contained within the Bladder is relieved from the distal end 2 into the proximal end (FIG. 7A). In the open position, the distal end of the Bladder is filled (FIG. 7B). The Bladder can be made of a non-compliant or semi-compliant material. It can be filled with sterile saline, air or other sterile fluid. Either or both Cutting and Spreading Tips can contain a Tissue Engagement Section 3.

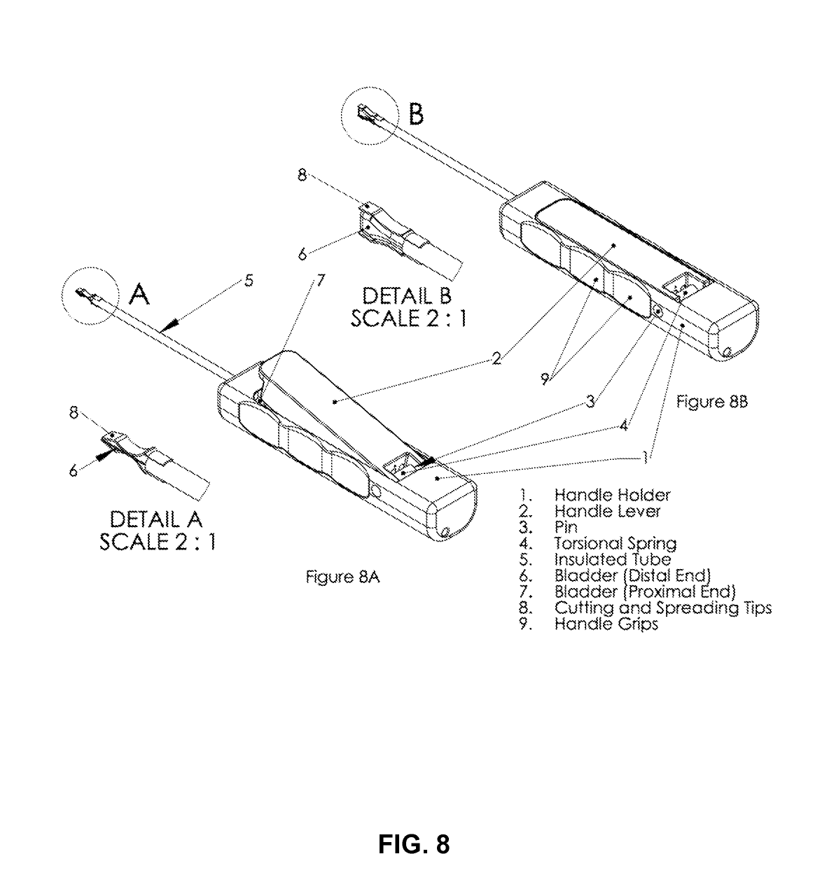

FIGS. 8A-B show another exemplary embodiment of a tip design for the bladder actuated tip tool according to invention. When the Handle Lever 2 is open, the Cutting and Spreading Tips 8 are closed (FIG. 8A). When the Handle Lever is depressed, the Cutting and Spreading Tips open (FIG. 8B). This handle is composed of a Handle Holder 1, a Handle Lever 2, and a Pin 3 holding the Handle Lever and Handle Holder together. A single Bladder 6, 7 extends from a chamber in the Handle Holder 1 through the Insulated Tube 5 and into the distal end of the Cutting and Spreading Tips. On the Pin, is a Torsional Spring 4, which holds the Handle Lever 2 open when in a relaxed position, keeping the Bladder at the distal end 6 deflated and inflated at the proximal end 7, thus bringing the Cutting and Spreading Tips 8 to a closed position (FIG. 8A). When the Handle Lever is depressed, the distal end of the Bladder 6 fills, causing the Cutting and Spreading Tips to open (FIG. 8B). Handle Grips 9 improve the ergonomics of design. This configuration can be made monopolar by insulating the tool, exposing one section of the Cutting and Spreading Tips 8 to create a cutting surface. As an alternative option, the tool can be made bipolar by insulating each Cutting and Spreading Tip from each other, and exposing surfaces on both tips to create either a cutting or coagulation feature.

What is claimed is:

1. A dual-purpose device for laparascopically performing a surgical procedure comprising a tip design, wherein said tip design comprises:
(a) an outer tube;
(b) a rod movable with said outer tube; and
(c) a flexure mechanism wherein a first part of said flexure mechanism is connected to said outer tube and a second part of said flexure mechanism connected to said rod, wherein said second part of said flexure mechanism forms a tissue engagement section at its distal end having two flat facing areas that can be separated from each other when said rod is movably engaged with said outer tube, wherein said separation separates tissue, and wherein the tip of at least part of said second part of said flexure mechanism is a cutting tip for cutting tissue.
